(19) United States
(12) Patent Application Publication
(10) Pub. No.: US 2008/0058921 A1
(43) Pub. Date: Mar. 6, 2008

(54) IMPROVED ADHESION OF A POLYMERIC COATING OF A DRUG ELUTING STENT

(76) Inventor: Jeffrey S. Lindquist, Maple Grove, MN (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/463,482

(22) Filed: Aug. 9, 2006

(51) Int. Cl. A61F 2/06

(52) U.S. Cl. 623/1.42

(57) ABSTRACT

An apparatus and method related to a drug eluting stent with improved adhesion between a drug excipient coating and a stent substrate is described. In one embodiment of the present invention, an apparatus comprises a stent substrate formed of a metal and/or a polymer and having a surface modified by exposure to ultraviolet light and an atomic oxygen molecule. A polymeric material is coupled with the surface of the stent substrate. In another embodiment, a method includes providing a stent with an adhesive property that is associated with the surface of the stent. The adhesive property of the stent is modified by exposing the surface to ultraviolet light and an atomic oxygen molecule.

### FIELD OF INVENTION

The present invention relates generally to a drug eluting stent, and in particular, but not by way of limitation, to surface preparation of a stent substrate of a drug eluting stent.

### BACKGROUND

Stents and stent delivery assemblies are utilized in a number of medical procedures and situations, and as such their structure and function are well known. A stent is a generally cylindrical prosthesis introduced via a catheter into a lumen of a body vessel in a configuration having generally reduced diameter and then expanded to the diameter of the vessel. In its expanded configuration, the stent supports and reinforces the vessel walls while maintaining the vessel in an open, unobstructed condition. In many applications stents are coated with a drug excipient coating that can be configured to release, for example, a pharmacological agent into tissue surrounding the stent.

Current drug-eluting stent (“DES”) coating technology often involves application of a solvated polymer blend onto a bare surface of a stent substrate using, for example, a spray application process. The level of adhesion of a drug excipient coating to a stent substrate of a DES is an important, and often overlooked, aspect of the DES. The level of bonding between the stent substrate and the polymeric coating can be, for example, a factor in kinetic drug release rate, product consistency, product safety, and device withdrawal resistance. Improved, consistent adhesion can also prevent coating adhesion related defects (e.g., coating lift, undercutting, holes, particulate formation, etc.) that can occur, in particular, in high strain areas of the DES upon expansion and/or crimping and can adversely affect, for example, drug release rate. Thus, a need exists for an apparatus and a method that provide a DES with enhanced adhesion between the drug excipient coating and the stent substrate.

### SUMMARY OF THE INVENTION

In one embodiment, an apparatus comprises a stent substrate formed of a metal and/or a polymer and having a surface modified by exposure to ultraviolet light and an atomic oxygen molecule. A polymeric material is coupled with the surface of the stent substrate. In another embodiment, a method includes providing a stent with an adhesive property that is associated with the surface of the stent. The adhesive property of the stent is modified by exposing the surface to ultraviolet light and an atomic oxygen molecule.

### BRIEF DESCRIPTION OF THE DRAWINGS

FIG. 1 is a schematic illustration of a DES that includes a drug excipient coating applied to a surface of a stent substrate after the stent substrate was exposed to an ultraviolet ozone (UV-ozone) process, according to an embodiment of the invention.

FIG. 2A illustrates a diagram of a DES in a contracted state, according to an embodiment of the invention.

FIG. 2B illustrates an enlarged view of an apex region from the DES shown in FIG. 2A, according to an embodiment of the invention.

FIG. 2C illustrates a cross-section of the apex region shown in FIG. 2B, according to an embodiment of the invention.

FIG. 3A illustrates a diagram of a DES in an expanded state, according to an embodiment of the invention.

FIG. 3B illustrates an enlarged view of an apex region from the DES shown in FIG. 3A, according to an embodiment of the invention.

FIG. 3C illustrates a cross-section of the apex region shown in FIG. 3B, according to an embodiment of the invention.

FIG. 4A shows a DES with a drug excipient coating that was applied to a bare metal substrate after the bare metal substrate was exposed to a UV-ozone process, according to an embodiment of the invention.

FIG. 4B illustrates a cross-section of one of the struts shown in FIG. 4A, according to an embodiment of the invention.

FIG. 5 illustrates a method for producing a DES that includes exposing a stent substrate of the DES to a UV-ozone process, according to an embodiment of the invention.

### DETAILED DESCRIPTION

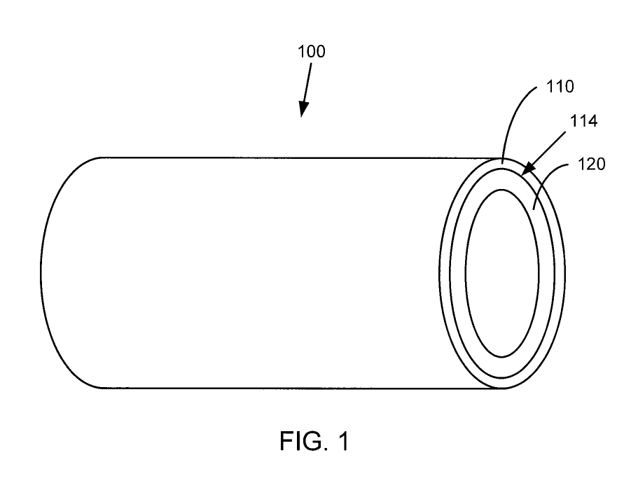

FIG. 1 illustrates a DES 100 that includes a drug excipient coating 110 applied to a surface 114 of a stent substrate 120. The drug excipient coating 110 and stent substrate 120 are configured such that the DES 100 can be inserted into a body lumen and a drug can be delivered from the drug excipient coating 110 to prevent, for example, thrombosis. The surface 114 of the stent substrate 120 has been exposed to an ultraviolet light and atomic oxygen in an ultraviolet-ozone (UV-ozone) process that promotes the adhesion of the drug excipient coating 110 to the surface 114 of the stent substrate 120. In particular, the UV-ozone process promotes the adhesion of the drug excipient coating 110 to the stent substrate 120 even when the DES 100 expands and contracts during normal use. The DES 100 can change, for example, from a contracted state to an expanded state when being inserted into a body lumen using a balloon catheter insertion technique.

The stent substrate 120 is a hollow-tubed structure that can be formed with interconnected or interwoven members that can be referred to as struts. The struts can be, for example, straight, serpentine, sinusoidal, or other shapes that allow the stent substrate 120 to expand from a reduced diameter (i.e., contracted state) to a diameter useful in a particular application (i.e., expanded state). Suitable materials for the stent substrate 120 include, for example, metals and alloys based on titanium (such as nitinol, nickel titanium alloys, thermo-memory allow materials), stainless steel, tantalum, nickel-chrome, clad composite filaments, polymers, co-polymers, or certain cobalt alloys including cobalt-chromium-nickel alloys.

The drug excipient coating 110 can be any type of appropriate combination of one or more biologically active materials (e.g., drug) and/or vehicles for the drug. The drug can be, for example, a genetic material, a pharmaceutical agent, a cell, an inhibitor, a non-genetic therapeutic agent, a polymer matrix having a therapeutic component or any other substance which would be desirable to deliver into a body lumen. The vehicle can be a binder, filler, disintegrant, lubricant, or coating that can include, for example, an inert polymeric material/compound.

Suitable polymeric materials that can serve as the vehicle for the drug within the drug excipient coating 110 include, for example, polyurethane and its copolymers, silicone and its copolymers, ethylene vinyl-acetate, polyethylene terephtalate, thermoplasic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins. In the illustrated embodiment, the drug excipient coating 110 is a styrene-isobutylene-styrene (SIBS) based coating (i.e., excipient) that is applied to the surface 114 of the substrate 120.

The drug excipient coating 110 can be applied to the surface 114 of the stent substrate 120 using any appropriate coating technique. For example, a solvated polymer blend (e.g., polymer blend that includes a solvent) with a therapeutic agent can be used as the drug excipient coating 110 and can be applied through direct spray application (e.g., spray-coating technique) onto the surface 114 of the stent substrate 120 after the surface 114 has been exposed to the UV-ozone process. In some embodiments, the drug excipient coating 110 can be applied to the stent substrate 120 by a micro-drop application technique, a roll-coating technique, and/or by dipping the stent substrate 120 into a solvated form of the drug excipient coating 110.

The surface 114 of the stent substrate 120 has been exposed to a UV-ozone process to promote the adhesion of the drug excipient coating 110 to the surface 114 of the stent substrate 120. The UV-ozone process is employed to modify an adhesive property associated with the surface 114 of the stent substrate 120. For example, the UV-ozone process can modify a chemical property of the stent substrate 120. Specifically, the UV-ozone process can add one or more oxygen-containing functional groups to the surface 114 of the stent substrate 120. These functional groups can modify the surface energy and/or morphology of the surface 114 of the stent substrate 120 to promote adhesion. The UV-ozone process can also remove organic contaminants (e.g., machine oils, human sebum, solder flux, etc.) and/or inorganic compounds (e.g., dusts, metal powder, quartz, etc.) from the surface 114 of the stent substrate 120 that would otherwise decrease and/or prevent adhesion of the drug excipient coating 110 to the surface 114.

The UV-ozone process can remove contaminants that occupy and/or block molecular bonds at the surface 114 that can then be allowed to molecularly bond to the drug excipient coating 110.

In the UV-ozone process, contaminants on the surface 114 of the stent substrate 120 are converted into volatile substances after being decomposed by UV light (e.g., UV rays) and oxidized by an atomic oxygen molecule. UV light of approximately 185 nm and 254 nm can be used to form and decompose ozone molecules, respectively, to produce the atomic oxygen from reactants used in the UV-ozone process. The atomic oxygen and/or ozone used in the UV-ozone process can be derived from an oxygen containing reactant such as oxygen (O₂). The UV light can be produced using, for example, a low-pressure quartz-mercury vapor lamp.

In many embodiments, the conditions of the UV-ozone process (e.g., reactant concentrations and/or types, length of exposure, temperature, pressure) can be adjusted and/or determined based on, for example, the type of drug excipient coating being applied to the stent substrate, type of application of the DES, and physical characteristics of the stent substrate. In some embodiments, for example, the stent substrate is exposed to the UV-ozone process at room temperature and pressure, but in several implementations, the UV-ozone process is conducted at different conditions such as, for example, elevated temperatures and/or pressures.

In many embodiments, the stent substrate 120 is exposed to the UV-ozone process for less than twenty minutes, but in some embodiments, the length of the exposure to the UV-ozone process can be adjusted (e.g., extended or shortened). The length of exposure, in some embodiments, is modified based on measurements of the cleanliness of the surface of the stent substrate. For example, the length of the UV-ozone process can be extended if water contact angles measured on the surface of the stent substrate are, for example, above a specified threshold value. The threshold value can be specified based on, for example, a correlation of adhesion characteristics of the stent substrate and water contact angles. The adhesive characteristics of the stent substrate can be measured using, for example, clinical scrub tests, pulsatile fatigue tests and/or peel adhesion evaluations.

Exposure to the UV-ozone process, in some embodiments, can be conducted in stages and/or at multiple sets of conditions. For example, the UV-ozone process can be conducted first at low temperature and later at high temperature with or without intervening processing such as electropolishing. In some embodiments only a portion or critical portions (e.g., apex regions) of the stent substrate 120 are exposed to the UV-ozone process using, for example, directional UV light and/or a directional introduction of reactants.

In some embodiments, the stent substrate 120 is cleaned using a preliminary cleaning before the surface 114 is exposed to the UV-ozone process. The preliminary cleaning can be conducted using, for example, an ultrasonic bath with a mild detergent or can be accomplished by, for example, scrubbing the surface of the stent substrate 120 with a brush. A primer coating, in some implementations, can be applied to the stent substrate 120 after it has been exposed to the UV-ozone process.

Each of the steps involved in producing the DES 100 can performed in a single chamber and/or multiple chambers. For example, a preliminary cleaning, if included in a particular DES 100 production flow, can be performed in a different chamber than the UV-ozone exposure and/or application of the drug excipient coating 110. Furthermore, the DES 100 can be produced using a process that includes batch processing and/or continuous processing steps.

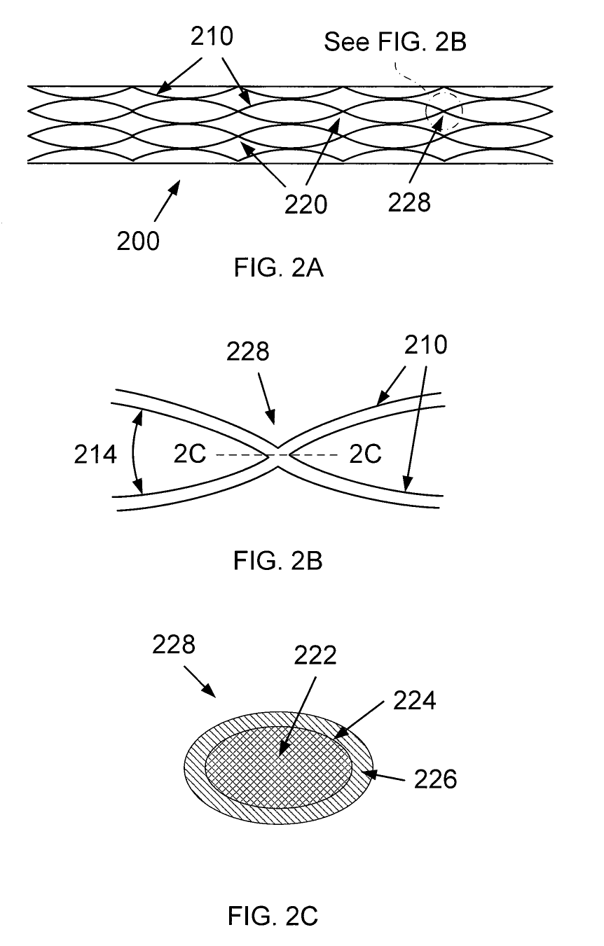

FIGS. 2A-C and 3A-C show a DES 200 with a metal stent substrate and drug excipient coating in a contracted and an expanded state, respectively. The bare surface of the metal stent substrate is exposed to a UV-ozone process, before the drug excipient coating is applied, to promote adhesion of the drug excipient coating to the metal stent substrate. Note that like or similar elements within FIGS. 2A-C and 3A-C are designated with identical reference numerals throughout the several views. In some embodiments, a polymer-based stent substrate, rather than a metal-based stent substrate, can be used.

The DES 200 shown in FIG. 2A includes a metal stent substrate formed from a framework of struts 210 that are coated with a drug excipient coating (e.g., SIBS based coating). Apex regions 220 are formed where one or more struts 210 meet to form the framework of the DES 200. The metal stent substrate is coated with the drug excipient coating while in the contracted state shown in FIG. 2A and after being exposed to a UV-ozone process. Because the metal stent substrate is formed in the contracted state, the DES 200 maintains its shape as shown in FIG. 2A without the application of external forces.

As shown in FIG. 2B, which is an enlarged view from FIG. 2A of an apex region 228, the struts 210 form an angle 214 that is acute when the DES 200 is in the contracted state. The apex region is an area of that is susceptible to tension and compression strain and/or deformation that can cause shearing forces that can, for example, separate the drug excipient coating from the surface of the metal stent substrate.

FIG. 2C illustrates a cross-section of apex region 228 from FIG. 2B (cut at line 2C) that shows the drug excipient coating 226 conformally coating a surface 224 of the metal stent substrate 222. Because the drug excipient coating 226 is applied to the surface 224 while in the contracted state, which is the rest state of the stent, shearing forces, for example, are generally not experienced by the drug excipient coating 226 at the surface 224 of the metal stent substrate 222.

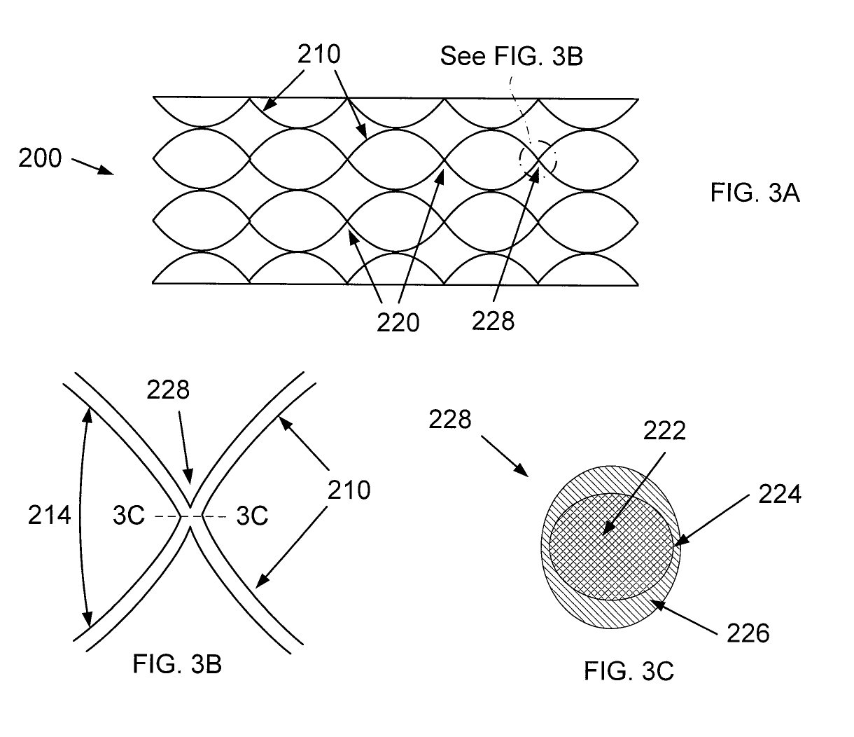

FIG. 3A shows the DES 200 in the same orientation as it was shown in FIG. 2A, but in the expanded rather than contracted state. FIG. 3A shows the struts 210 and apex regions 220 that form the framework of the DES 200. FIG. 3B, which is an enlarged view of apex region 228 shown in FIG. 3A (and the same apex region as shown in FIG. 2B), shows that the struts 210 form an angle 214 that is obtuse when the DES 200 is in the expanded state.

FIG. 3C, which is a cross-section of apex region 228 from FIG. 3B (cut at line 3C), shows the drug excipient coating 226 conformally coating a surface 224 of the metal stent substrate 222. Because the drug excipient coating 226 is applied to the surface 224 while in the contracted state, shearing forces are experienced by the drug excipient coating 226 at the surface 224 of the metal stent substrate 222 when the DES 200 is in the expanded state. Although shearing forces can exist at any point between the metal stent substrate 222 and the drug excipient coating 226, strains and stresses can be relatively significant in the apex region 228. The UV-ozone process can promote adhesion of the drug excipient coating 226 to the metal stent substrate 222 when these shearing forces exist. The UV-ozone process can also promote adhesion of the drug excipient coating 226 to the metal stent substrate 222 when exposed to, for example, abrasive environments that strain the drug excipient coating 226.

In many embodiments, the metal stent substrate 222 is coated with the drug excipient coating 226 in the expanded state rather than the contracted state. In some embodiments, the drug excipient coating 226 is applied to the metal stent substrate 222 in an intermediate state of contraction and/or expansion that is the result of the application of some external force (e.g., intentionally applied external force).

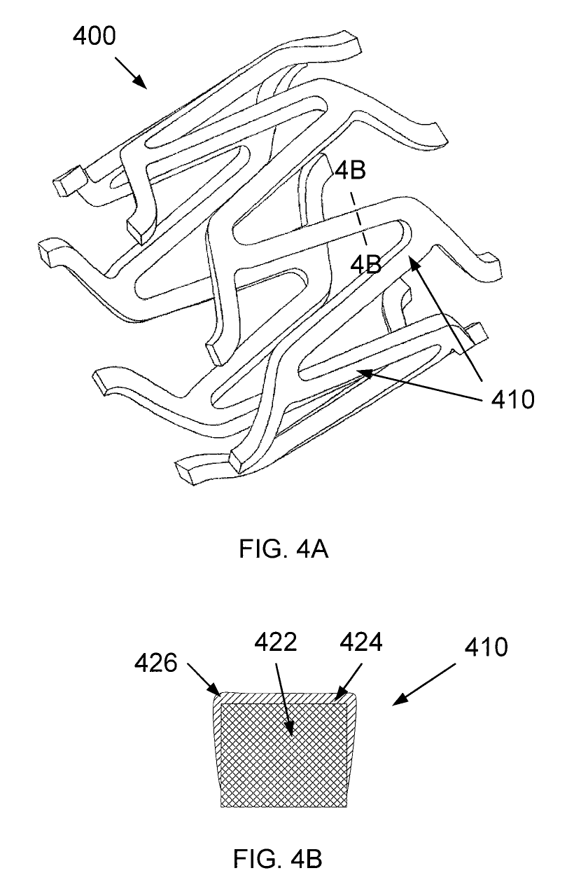

FIG. 4A illustrates a DES 400 with struts 410 that form the framework of the DES 400. The DES 400 is coated with a drug excipient coating that is applied to a bare metal substrate of the DES after the bare metal substrate is exposed to a UV-ozone process. The exposure of the bare metal substrate to UV light and atomic oxygen promotes adhesion of the drug excipient coating to the bare metal substrate. In this embodiment, only a portion of the surface of the DES 400 is coated with the drug excipient coating. In some embodiments, a polymer-based stent substrate, rather than a metal-based stent substrate, can be used.

FIG. 4B illustrates a cross-section of one of the struts 410 shown in FIG. 4A (cut at line 4B). FIG. 4B shows that the drug excipient coating 426, rather than conformally coating the surface 424 of the metal stent substrate 422, covers a portion of the surface 424 of the metal stent substrate 422.

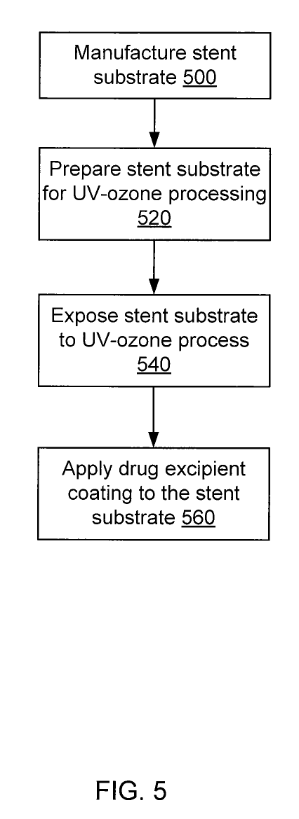

Referring now to FIG. 5, it illustrates a method for producing a DES that includes exposing a stent substrate (e.g., metal stent substrate and/or polymer stent substrate) of the DES to a UV-ozone process. The DES includes a stent substrate and drug excipient coating applied to the surface of the stent substrate. In this exemplary embodiment, the stent substrate is first manufactured 500 using, for example, a laser-cutting technique to cut the stent substrate from stainless steel. The stent substrate can be cut into any pattern or shape that will be useful for the target application. In some embodiments, the stent is cut into a pattern (e.g., strut type) that will promote the effectiveness of the UV-ozone process.

After the stent substrate has been manufactured 500, the stent substrate is prepared for UV-ozone processing 520. For example, the surface of the stent substrate can be roughened to promote adhesion of the drug excipient coating that will later be applied to the surface. The stent substrate can also, in several embodiments, be cleaned using, for example, conventional cleaning techniques (e.g., brush scrubbing) to remove compounds and/or residuals such as inorganic salts that may not be effectively removed from the surface of the stent substrate by some UV-ozone processes. In some embodiments for example, the preparation of the stent substrate for UV-ozone processing 520 includes preparing the stent substrate by, for example, cleaning with a detergent. Some of these compounds and/or residuals on the surface can be a result of, for example, handling of the stent substrate or the process used to manufacture the stent substrate at 500.

The stent substrate is then exposed to the UV-ozone process 540 in, for example, a UV-ozone processing chamber. The conditions of the UV-ozone process (e.g., reactant concentrations and/or types, length of exposure, temperature, pressure) can be specified based on the type of drug excipient coating being applied to the stent substrate, type of application of the DES, and the physical characteristics of the stent substrate. The conditions of the preparation of the stent substrate 520 and the exposure to the UV-ozone process 540 can be optimized to produce a particular level of surface cleanliness measured using, for example, water contact angles.

After the stent substrate has been exposed to the UV-ozone process at 540, a drug excipient coating is applied to the stent substrate 560. For example, a solvated polymer blend (e.g., SIBS) with a pharmacological agent can be used as the drug excipient coating and can be applied to the surface of the stent subsrate through direct spray application. In some embodiments, the drug excipient coating can be applied to the stent substrate using, for example, a roll-coating technique, a micro-drop application technique, and/or a dipping technique.

In conclusion, the present invention is related to a DES with a stent substrate that has been exposed to a UV-ozone process. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. A method, comprising:
providing a stent having a surface, the surface having an adhesive property; and
modifying the adhesive property associated with the surface of the stent by exposing the surface to ultraviolet light and an atomic oxygen molecule.

2. The method of claim 1, further comprising coating the surface with a polymeric material.

3. The method of claim 1, further comprising coating the surface with a polymeric material, the polymeric material including a polymer and at least one of a biologically active material or a solvent.

4. The method of claim 1, further comprising coating the surface with a styrene-isobutylene-styrene (SIBS) and at least one of a biologically active material or a solvent.

5. The method of claim 1, further comprising coating the surface with a polymeric material using at least one of a spray-coating technique, a roll-coating technique, a micro-drop technique, or a dipping technique.

6. The method of claim 1, wherein the adhesive property includes at least one of a surface energy of the surface or a morphology of the surface.

7. The method of claim 1, wherein the modifying includes removing contaminants.

8. The method of claim 1, wherein at least a portion of the surface of the stent is formed of at least one of a metal or a polymer.

9. The method of claim 1, wherein the atomic oxygen molecule is derived from ozone.

10. The method of claim 1, wherein the modifying includes modifying a portion of the surface of the stent.

11. The method of claim 1, wherein at least a portion of the surface is on the outside of the stent.

12. An apparatus, comprising:
a stent substrate formed of at least one of a metal or a polymer and having a surface modified by exposure to ultraviolet light and an atomic oxygen molecule; and
a drug excipient material coupled with the surface of the stent substrate.

13. The apparatus of claim 12, wherein the surface has an adhesive property associated with a molecular bond, the adhesive property is modified by modifying the surface.

14. The apparatus of claim 12, wherein the surface has an adhesive property, the adhesive property of the surface is modified by removing contaminants.

15. The apparatus of claim 12, wherein the drug excipient material is coupled via molecular bonding with the surface of the stent substrate.

16. The apparatus of claim 12, wherein the drug excipient material is mechanically coupled to the surface of the stent substrate.

17. The apparatus of claim 12, wherein the surface is substantially an outside surface of the stent substrate.

18. The apparatus of claim 12, wherein the drug excipient material includes a polymer and at least one of a biologically active material or a solvent.

19. The apparatus of claim 12, wherein the drug excipient material includes styrene-isobutylene-styrene (SIBS) and at least one of a biologically active material or a solvent.

20. The apparatus of claim 12, wherein the atomic oxygen molecule is derived from ozone.

21. A method, comprising:
providing a stent having a surface, at least a portion of the stent being formed of at least one of a metal or a polymer; and
exposing the surface to ultraviolet light and an atomic oxygen molecule.

22. The method of claim 21, wherein the exposing modifies an adhesive property associated with the surface of the stent, the adhesive property is associated with at least one of a surface energy of the surface or a morphology of the surface.

23. The method of claim 21, wherein the exposing modifies an adhesive property associated with the surface of the stent.

24. The method of claim 21, further comprising coating the surface with a drug excipient material, the exposing modifies an adhesive property associated with the surface of the stent, the adhesive property is associated with the drug excipient material.

25. The method of claim 21, further comprising coating the surface with a polymeric material.

26. The method of claim 21, further comprising coating the surface with a polymeric material, the polymeric material including a polymer and at least one of a biologically active material or a solvent.

27. The method of claim 21, further comprising coating the surface with a styrene-isobutylene-styrene (SIBS) and at least one of a biologically active material or a solvent.

28. The method of claim 21, further comprising coating the surface with a polymeric material by at least one of spraying the polymeric material onto the surface of the stent or dipping the stent into the polymeric material.

29. The method of claim 21, wherein the exposing removes contaminants from the surface of the stent.

30. The method of claim 21, wherein the atomic oxygen molecule is derived from ozone.

31. The method of claim 21, further comprising coating the surface with a polymeric material, the exposing occurs in a chamber, the coating occurs in the chamber.

32. The method of claim 21, wherein the surface is substantially an outside surface of the stent.
